(19) United States
(12) Patent Application Publication
(10) Pub. No.: US 2024/0295049 A1
(43) Pub. Date: Sep. 5, 2024

(54) A DEVICE AND A METHOD

(71) Applicant: Imperial College Innovations Limited, London (GB)

(72) Inventors: Guang-Zhong Yang, London (GB); Mohamed Ek Abdelaziz, London (GB); Burak Temelkuran, London (GB); Hyun-Taek Lee, London (GB); Jinshi Zhao, London (GB); Florent Seichepine, London (GB)

(21) Appl. No.: 18/258,870

(22) PCT Filed: Dec. 22, 2021
(86) PCT No.: PCT/GB2021/053406

§ 371 Date: Jun. 22, 2023

(30) Foreign Application Priority Data
Dec. 22, 2020 (GB) 2020346.9

(51) Int. Cl. D01D 5/24; B29C 55/22; B29L 23/00; D01F 8/00; H05B 3/56

(52) U.S. Cl. CPC D01D 5/24; B29C 55/22; D01F 8/00; H05B 3/56; B29L 2023/007; H05B 2203/017

(57) ABSTRACT

A device comprising a bendable fibre comprising a fibre axis, a thermally expandable material and a resistance wire extending longitudinally through the fibre spaced apart from the fibre axis; wherein, in use, electrical power applied to the resistance wire causes the temperature of the resistance wire to increase.

The present application relates generally to a device formed of a fibre that is both bendable and actuatable and a method of manufacturing such a fibre.

The invention may have application in the field of minimally invasive surgery (MIS). MIS procedures pose several challenges to surgeons as they are required to work in small, narrow and/or tortuous spaces inside the body. Small diameter devices, like fibres or scopes, are required to access the specific site that a procedure is to be performed while minimizing the size of the incision on the body of the patient. Further, surgeons require the ability to steer a functional tip of the fibre with precise control towards a target area in order to perform therapeutic or diagnostic techniques. The steering may be controlled by a sequence (or robotic control) that allows a surgeon to perform a controlled scanning of a suspicious area (such as a tumour or tumour bed after removal of a tumour) to detect and ablate diseases.

The invention may also have application in the field of soft robotics. Soft robots are made from soft, elastic materials and offer unique opportunities in areas in which conventional rigid robots are not viable such as drug delivery, non-invasive surgical procedures, assistive devices, or artificial organs.

There are various approaches to provide actuation of soft robots that include a pneumatic mechanism powered by compressed gas or vacuum and a tendon/pull-wire/cable-driven mechanism. However, each of these approaches requires a connection to a bulky control and drive system which provides limitations to making compact and lightweight autonomous robotic systems.

Dielectric elastomer actuators (DEAs) are an attractive option for soft robotic applications, but they require relatively high-actuation voltages which limit their usability in human environments.

Known thermal drawing processes start with a preform that is a block of material, from which fibres can be drawn. A preform generally comprises a substantially cylindrical structure, although a preform may comprise any shape.

A longitudinal direction, also referred to herein as the draw direction, can be defined as extending through the length of the preform in the direction along which a fibre would be drawn. A radial direction can be defined as extending radially outwardly from, and perpendicular to, an axis extending in the longitudinal direction (longitudinal axis).

Preforms have a larger transverse cross-sectional area than the fibres which are drawn from them and the resultant fibre which can be drawn from a single preform can be, for example, one thousand times longer than the original preform. During the drawing of a fibre from a preform, the structure shrinks in the transverse direction of the preform and is elongated in the longitudinal direction (the draw direction). As such, the precise structure of the preform in terms of its composition, its shape, its size and any other features has a significant impact on the resulting fibre.

Preforms may be fabricated by one of, or a combination of, a number of techniques including: hot press, cast-moulding or injection moulding of thermoplastic pellets in vacuum; additive manufacturing techniques; direct machining of commercially acquired rods or bars; and rolling of thermoplastic sheets/films and consolidating into preforms.

The process of drawing a fibre comprises heating a portion of the preform to a temperature over its glass transition temperature which in turn allows the neck-down drawing of the preform to take place. During this process, the viscosity of the preform may decrease several orders of magnitude and the preform may be drawn down under its own weight. The drawing temperature should be primarily selected based on the preform material to be used. The higher the glass transition temperature of the preform material to be used, the higher the required draw temperature. It will be appreciated that heating of the preform may be provided by the draw apparatus such as by a resistive heater comprising part of the draw apparatus. Alternatively, heating of the preform may be provided by an external heating apparatus such as a resistive heater, a furnace configured to surround the portion of the preform to be drawn, or any other suitable apparatus. After the drawing of a portion of the preform into a fibre, the resultant fibre may cool (may be quenched) in order to set the shape of the fibre.

Cooling of the fibre may occur in ambient conditions as a result of removing the fibre from the influence of the heating apparatus without further need for cooling. Alternatively, the fibre may pass through a region cooled by a cooling apparatus either forming part of the draw apparatus or independently provided. The cooling apparatus may comprise a cooled enclosure through which the drawn fibre passes or may comprise an apparatus for providing cooled air over the fibre. Any suitable method for providing cooling of the fibre may be used. In addition, prior to heating the preform for drawing the fibre therefrom, a pre-heating stage may be provided. Preheating may be performed by any suitable heating apparatus and may be performed in order to avoid thermal shock and to help achieve a better temperature uniformity. It will be appreciated that in some examples, preheating may be unnecessary or may comprise part of the heating step.

The drawing of the fibre may be monitored by a monitoring apparatus. The monitoring apparatus may be a laser micrometre for measuring the diameter of the fibre and/or a tension sensor for measuring the effective tension on the fibre.

During the drawing of the preform into a fibre, the temperature profile, the down-feed speed and the draw speed may be adjusted. The temperature profile describes the temperature regions through which the preform and resultant fibre pass. The down-feed speed is the rate at which the preform is moved into the heated region. The draw speed is the rate at which the fibre is pulled through the draw apparatus. By varying the temperature profile, the down-feed speed and the draw speed, the thickness of the resultant fibre may be adjusted.

According to a first aspect of the invention, there is provided a device comprising a bendable fibre comprising a fibre axis, a thermally expandable material and a resistance wire extending longitudinally through the fibre spaced apart from the fibre axis; wherein, in use, electrical power applied to the resistance wire causes the temperature of the resistance wire to increase.

The bendable fibre may be a fibre with a high aspect ratio, that is a fibre with a small width/diameter (e.g. 1-5 mm) and a large length (e.g. 0.2-1 m). The bendable fibre may also comprise materials that are flexible/have a low stiffness so that it may bend readily and repeatably. The device may therefore be suitable for use in minimally invasive surgical applications, for example, in which the bendable fibre is required to enter narrow and tortuous environments in the body, such as the lungs, arteries or intestinal tracts for example.

The resistance wire extending longitudinally through the fibre may have a high electrical resistance meaning that applying electrical power to the resistance wire may cause it to increase in temperature. When the resistance wire is heated it may cause thermally expandable material proximal to the resistance wire to heat also. Accordingly, the thermally expandable material proximal to the resistance wire may expand due to increasing in temperature while thermally expandable material further away from the resistance wire may expand less or not at all. As the resistance wire is apart from the axis of the fibre, the expansion of thermally expandable material will be asymmetric across the cross-section of the fibre.

Due to the high aspect ratio of the fibre, expansion of the thermally expandable material may affect the dimensions of the fibre primarily in the longitudinal direction (i.e. along the length of the fibre). As thermally expandable material proximal to the resistance wire becomes longer than thermally expandable material further away from the resistance, the bendable fibre may be caused to curl or deflect.

Therefore, by means of the invention a device is provided comprising a fibre that may be caused to deflect by applying an electrical current to the resistance wire.

In embodiments of the invention the resistance wire may comprise a material with high electrical resistivity, such as stainless-steel alloys. High electrical resistivity may provide the resistance wire with a higher electrical resistance and therefore increase the degree to which the resistance wire is heated by applying a current through it. The hotter the resistance wire, the hotter the material that surrounds it may become and the more it may expand. Hence, a resistance wire material with high electrical resistivity may facilitate more acute deflection of the fibre.

In embodiments of the invention, the thermally expandable material may have a coefficient of linear thermal expansion between 0.2×10⁻⁴ and 10×10⁻⁴ K⁻¹.

In such embodiments of the invention, the thermally expandable material may be a polymer such as polycarbonate (PC), polysulfone (PSU), polyethylenimine (PEI) or poly(methyl methacrylate) (PMMA), for example.

The higher the coefficient of linear thermal expansion of the thermally expandable material, the greater the degree to which the thermally expandable material will expand due to increasing in temperature. Therefore, a fibre comprising a thermally expandable material with a higher coefficient of linear thermal expansion may be caused to deflect more acutely for a given temperature increase.

In embodiments of the invention the fibre may comprise a material with a low heat transmission coefficient such as a polymer (PC, PSU, PEI or PMMA, for example). The low heat transmission coefficient may increase the potential temperature gradient across the cross-section of the fibre, thereby providing a greater localisation of material expansion which may, in turn, facilitate more acute deflection.

In embodiments of the invention, the resistance wire may comprise a material having a resistivity between 1×10⁻⁷ and 5×10⁻⁶ Ω·m at 20° C.

In such embodiments of the invention the resistance wire may comprise any suitable material with a resistivity between 1×10⁻⁷ and 5×10⁻⁶ Ω·m at 20° C., such as nichrome (resistivity of 1.135×10⁻⁶ Ω·m), stainless-steel (resistivity of 8.1×10⁻⁷ Ω·m) or nickel titanium (resistivity of 9.8×10⁻⁷ Ω·m) for example. The greater the resistivity of the material, the greater the resistance of the resistance wire and the more its temperature will increase when electrical power is applied to it. Hence, a resistance wire material with a greater resistivity may facilitate a greater degree of deflection of the fibre.

In embodiments of the invention the fibre may further comprise a void extending longitudinally at least partially through the fibre.

In such embodiments of the invention the void may extend substantially through the centre of the fibre and may therefore increase the distance that heat must travel through thermally expandable material in order to travel from one side of the fibre's cross section to an opposite side. The time required for heat to travel through the fibre may therefore increase such that a greater temperature gradient may develop across the cross-section of the fibre. There may also be a greater temperature gradient when the temperature of the fibre reaches steady state. In other words, for thermally expandable material in the fibre which has the void positioned between it and the resistance wire, the void acts to thermally insulate the thermally expandable material from the resistance wire so that a greater difference in temperature across the cross-section of the fibre may be achieved. The greater the difference in temperature of the thermally expandable material in two opposite parts of the fibre, the greater the resulting deflection of the fibre may be. Further, the larger the void is, and the thinner that that the thermally expandable material is, the greater the effect on temperature gradient across the fibre will be.

In another embodiment, the void may extend between the resistance wire and an outer surface of the fibre. Hence, rather than acting to insulate one side of the fibre from the other, the void may act to insulate the outer surface of the fibre from the resistance wire. This may be particularly advantageous if the device is for surgical applications where the outer surface of the fibre may touch internal body tissues of a patient.

A void extending through the fibre may also have application as a working channel when the device is in use in minimally invasive surgical procedures. The working channel (which may be a central lumen or side channel) may facilitate the transport of cooling fluids, drugs or bodily fluids. Also, an additional device, such as a catheter, probe or functionalised polymeric or glass fibre, may be deployed via the working channel. The device may therefore act as an actuation mechanism for actuating another device during a minimally invasive surgical procedure, for example.

In some embodiments of the invention, the fibre may comprise a plurality of voids, wherein each void may increase the temperature gradient across the fibre and, optionally, one or more of the voids may have application as a working channel.

In embodiments of the invention the device may further comprise an electrical conductor operatively connected to the resistance wire for applying electrical power to the resistance wire.

In such embodiments of the invention an electrical power source may be connected to the electrical conductor and an electrical circuit comprising the resistance wire, electrical conductor and electrical power source may be formed. An electrical current may flow through the electrical circuit and electrical power may thereby be applied to the resistance wire. The electrical circuit may be configured to include part or all of the resistance wire.

In embodiments of the invention the electrical conductor may comprise a conducting wire extending longitudinally through the fibre spaced apart from the resistance wire, which conducting wire is electrically connectable to the resistance wire.

In such embodiments of the invention the conducting wire may be electrically connected to the resistance wire, at either end of the fibre for example, and close a circuit loop so that an electrical current can travel along the resistance wire and then return along the conducting wire (or vice versa). The conducting wire may have a lower resistance than the resistance wire so that the conducting wire exhibits a low change in temperature, in use, compared to the resistance wire. This may prevent the conducting wire from causing a deflection of the fibre that might detract from the deflection caused by the resistance wire.

For example, the conducting wire may comprise a material with lower electrical resistivity than the material of the resistance wire (copper, silver or gold for example), have larger transverse dimensions than the resistance wire or be absent of surface etching that is present on the resistance wire.

Alternatively, the conducting wire may have a similar or equal resistance to the resistance wire. In use, the conducting would therefore increase in temperature similarly to the resistance wire, essentially acting as a second resistance wire as well as forming part of the electrical conductor. This may be particularly advantageous if the conducting wire is positioned close to the resistance wire so that the increase in temperature of both wires amplifies the temperature increase of the thermally expandable material proximal to the wires and increases the deflection of the fibre that may be caused.

In embodiments of the invention the electrical conductor may comprise a first conducting wire and a second conducting wire, each extending longitudinally through the fibre spaced apart from the resistance wire and from one another, and the first and second conducting wires are each electrically connectable to the resistance wire.

In such embodiments of the invention, the electrical conductor may form an electrical circuit wherein an electrical current may travel along the first conducting wire in a first direction through the fibre and along a combination of the resistance wire and second conducting wire in a second direction through the fibre substantially opposite to the first direction. Also, the first and second conducting wires may have a lower resistance than the resistance wire.

Therefore, in use, an electrical current may flow through only part of the resistance wire and only this part of the resistance wire will increase in temperature. Meanwhile, the rest of the resistance wire will exhibit minimal change in temperature as no current will flow through it and the conducting wires will also show relatively low change in temperature due to their lower electrical resistance. Accordingly, the temperature increase in thermally expandable material will be localised around the heated part of the resistance wire and the resulting deflection of the fibre will be similarly localised.

The portion of the fibre in which deflection occurs is therefore dependent on where, along the length of the fibre, the resistance wire is connected to the first and second conducting wires. For example, the fibre may comprise a proximal end and a distal end and in surgical applications it may be advantageous for a portion of the fibre positioned at the distal end of the fibre to be the only portion that deflects. In which case, the first conducting wire may be connected to the resistance wire at the distal end of the fibre and the second conducting wire may be connected to the resistance wire such that electrical current will only flow through the resistance wire extending through the relevant portion of the fibre.

In embodiments of the invention a portion of the resistance wire may comprise an etched surface.

In such embodiments of the invention the etched portion of the resistance wire may have an increased electrical resistance. In use, when electrical power is applied to the resistance wire, the etched portion may therefore exhibit a larger temperature increase causing greater local expansion of the thermally expandable material. The portion of the fibre through which the etched portion of the fibre extends may therefore exhibit a greater degree of deflection.

In embodiments of the invention the fibre may comprise a plurality of circumferential grooves positioned incrementally along at least a portion of the fibre.

In such embodiments of the invention the circumferential grooves may reduce the stiffness of the fibre and therefore increase its bendability in the portion in which the circumferential grooves are positioned. This may enable a greater degree of deflection of this portion of the fibre in response to the thermal expansion of the thermally expandable material without requiring an increase in the power supplied.

In embodiments of the invention the fibre may comprise a portion of the fibre has a smaller diameter than the rest of the fibre.

In such embodiments of the invention the reduced diameter may reduce the stiffness of that portion fibre and therefore increase the bendability of that portion of the fibre. This may enable a greater degree of deflection of this portion of the fibre in response to the thermal expansion of the thermally expandable material without requiring an increase in the power supplied.

In embodiments of the invention the fibre may comprise a first portion comprising a first thermally expandable material and a second portion comprising a second thermally expandable material, the first portion being more bendable than the second portion and/or the first thermally expandable material having a higher coefficient of linear thermal expansion than the second thermally expandable material.

In such embodiments of the invention the first portion may be more bendable than the second portion due the first thermally expandable material being softer, more flexible or have a lower stiffness than the second thermally expandable material. If the first portion is more bendable than the second portion then a greater degree of deflection may be possible in the first portion in response to the thermal expansion of the thermally expandable material without requiring an increase in the power supplied.

If the first thermally expandable material has a higher coefficient of linear thermal expansion then the first portion of the fibre may exhibit a greater amount of thermal expansion without requiring an increase in the power supplied and therefore a greater degree of deflection.

In embodiments of the invention the device may further comprise a restrictive sheath adapted to receive at least part of the fibre and restrict deflection of any part of the fibre received within the restrictive sheath.

In such embodiments of the invention, the restrictive sheath may be used to restrict deflection of a portion of the fibre received within the sheath while the rest of the fibre is free to deflect in response to thermal expansion caused by an electrical current.

In embodiments of the invention the fibre may further comprise a plurality of resistance wires, each extending longitudinally through the fibre and spaced apart from the fibre axis.

In such embodiments of the invention, electrical power may be applied to each resistance wire separately or in a variety of combinations to facilitate a variety of different deflections of the fibre. For example, the fibre may comprise two resistance wires extending longitudinally through the fibre and positioned substantially opposite to one another in the cross-section of the fibre. Such a fibre may be deflected in a first deflection by applying electrical power to the first resistance wire, and in a second direction opposite to the first direction by applying electrical power to the second resistance wire.

According to a second aspect of the invention, there is provided a method of manufacturing a bendable fibre comprising a fibre axis, thermally expandable material and a resistance wire spaced apart from the fibre axis using a draw apparatus, the method comprising the steps of: providing a preform comprising a preform axis, a thermally expandable material and a resistance wire channel extending longitudinally through the preform spaced apart from the preform axis, to the draw apparatus; heating a portion of the preform; drawing the heated portion of the preform in order to form a fibre; and feeding a resistance wire into the resistance wire channel during or after the draw such that the resistance wire is positioned within the resistance wire channel of the fibre spaced apart from the fibre axis.

In embodiments of the invention, the preform comprises a cross-section which is substantially maintained as the preform is drawn to form the fibre, i.e. the size, shape and position of the resistance wire channel remains constant relative to the preform/fibre. However, the actual dimensions that traverse the fibre cross-section are significantly smaller than those of the preform cross-section. It is therefore possible to provide a preform with a complex cross-sectional design that is relatively straightforward to produce at a large scale and then draw the preform to form the fibre with the same cross-sectional design at a scale that might otherwise be difficult or expensive to produce due to its intricacy, particularly in large volumes.

By means of the invention it is possible to use existing draw apparatuses to produce a fibre comprising a resistance wire such as that forming part of the first aspect of the invention. Hence, at relatively low cost a draw apparatus can be used to manufacture fibres comprising a resistance wire by employing the method defined above. This enables scalable manufacture of fibres with actuation capabilities that may be particularly advantageous, although not exclusively so, in the fields of minimally invasive surgical devices and soft robotics.

In embodiments of the invention, the preform may further comprise a first conducting wire channel, and the method comprises the further step of: feeding a first conducting wire into the first conducting wire channel during or after the draw.

In such embodiments of the invention the first conducting wire may be electrically connected to the resistance wire. The first conducting wire and resistance wire may be electrically connected by any suitable means and method. For example, the first conducting wire and resistance wire may each extend from an end of the fibre and be connected to one another outside of the fibre. In order for the first conducting wire and resistance wire to extend from an end of the fibre, thermally expandable material may be stripped from the wires at that end. In another example, the first conducting wire and resistance wire may each extend through the fibre to be flush with an end of the fibre and may be connected by applying solder or conductive ink paste to the end of the fibre. In a further example, the first conducting wire and resistance wire may be connected at a point along the length of the fibre by cutting into the fibre and soldering from a location on the first conducting wire to a location on the resistance wire or applying conductive ink paste.

In embodiments of the invention, the preform may further comprise a second conducting wire channel, and the method comprises the further step of: feeding a second conducting wire into the second conducting wire channel during or after the draw.

In such embodiments of the invention the second conducting wire may be electrically connected to the resistance wire, similarly to the first conducting wire, by any suitable means and method.

In embodiments of the invention, the method may comprise the further step of etching the surface of the resistance wire prior to feeding it into the resistance wire channel.

In such embodiments of the invention a fibre may be produced wherein one or more portions of the resistance wire comprises an etched surface, thereby varying the degree of deflection possible along the length of the fibre.

In embodiments of the invention, the method may comprise the further step of laser profiling a plurality of circumferential grooves positioned incrementally along a portion of the fibre.

In such embodiments of the invention a fibre may be produced wherein a portion of the fibre comprises circumferential grooves, thereby increasing the bendability of that portion of the fibre and varying the degree of deflection possible along the length of the fibre. In some embodiments of the invention the method may comprise laser profiling a plurality of circumferential grooves positioned incrementally along a plurality of portions of the fibre.

In embodiments of the invention, the step of drawing the heated portion of the preform may comprise varying the speed at which the heated portion of the preform is drawn in order to vary the diameter of the resulting fibre along its length.

In such embodiments of the invention a fibre may be produced wherein the diameter of the fibre varies along its length, thereby varying the degree of deflection possible along the length of the fibre.

In embodiments of the invention the preform may comprise a plurality of resistance wire channels, and the step of feeding the resistance wire into the resistance wire channel comprises feeding each of the plurality of resistance wires into a respective one of the plurality of resistance wire channels.

In such embodiments of the invention a fibre may be manufactured that comprises a plurality of resistance wires. A fibre may therefore be manufactured that has the ability to perform a number of different deflections depending on which of the resistance wires are caused to increase in temperature by applying an electrical current through them.

Also, the step of feeding each of the plurality of resistance wires into a respective one of the plurality of primary channels may comprise feeding each of the plurality of resistance wires from a respective one of a plurality of feeders into a proximal end of a respective one of the plurality of primary channels.

The steps of any method disclosed herein do not have to be performed in the exact order disclosed, unless explicitly stated or understood by the skilled person.

The invention will now be described by way of example only with reference to the accompanying drawings in which:

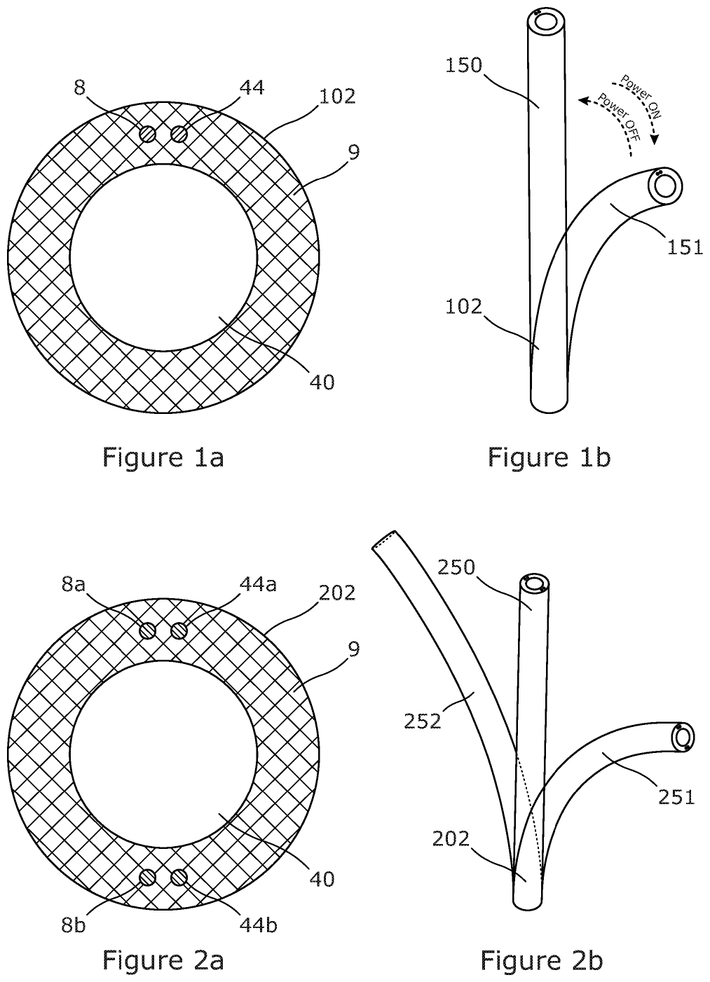

FIG. 1a is a cross-sectional schematic representation of a fibre according to an embodiment of the first aspect of the invention.

FIG. 1b is a schematic representation showing deflection of the fibre shown in FIG. 1a.

FIG. 2a is a cross-sectional schematic representation of a fibre according to a further embodiment of the first aspect of the invention.

FIG. 2b is a schematic representation showing deflection possible of the fibre shown in FIG. 2a.

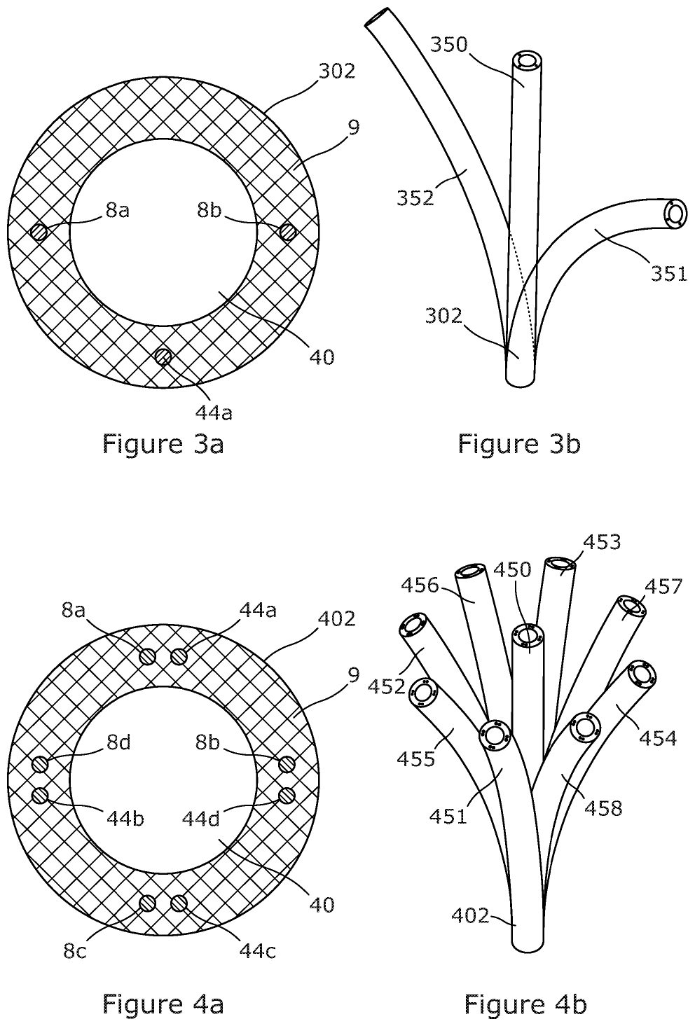

FIG. 3a is a cross-sectional schematic representation of a fibre according to a further embodiment of the first aspect of the invention.

FIG. 3b is a schematic representation showing deflection of the fibre shown in FIG. 3a.

FIG. 4a is a cross-sectional schematic representation of a fibre according to a further embodiment of the first aspect of the invention.

FIG. 4b is a schematic representation showing deflection of the fibre shown in FIG. 4a.

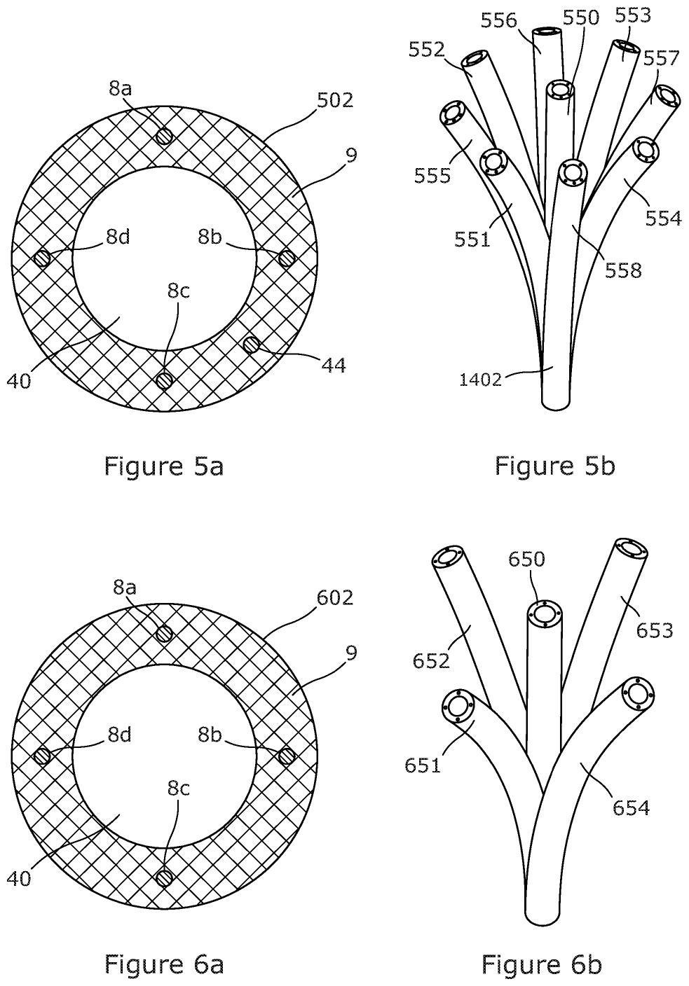

FIG. 5a is a cross-sectional schematic representation of a fibre according to a further embodiment of the first aspect of the invention.

FIG. 5b is a schematic representation showing deflection of the fibre shown in FIG. 5a.

FIG. 6a is a cross-sectional schematic representation of a fibre according to a further embodiment of the first aspect of the invention.

FIG. 6b is a schematic representation of actuation possible of the fibre shown in FIG. 6a.

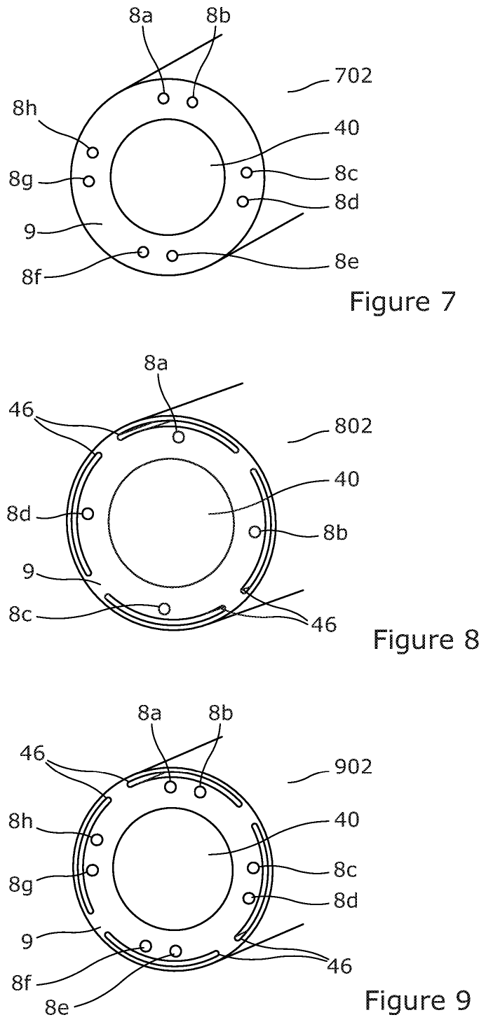

FIGS. 7, 8 and 9 are cross-sectional schematic representations of different fibres according to further embodiments of the first aspect of the invention.

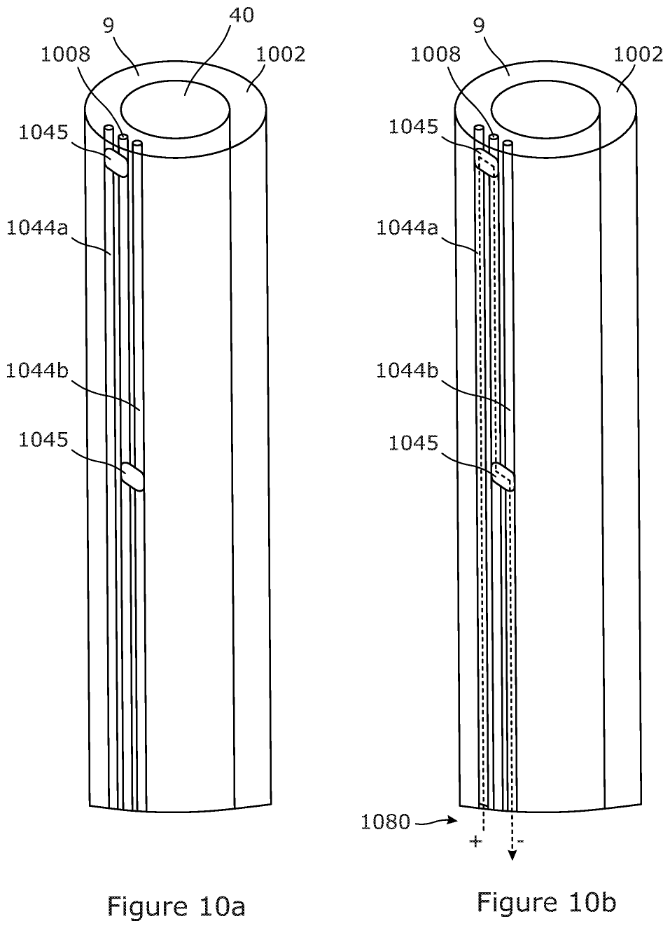

FIGS. 10a and 10b are schematic representation of a fibre according to a further embodiment of the first aspect of the invention.

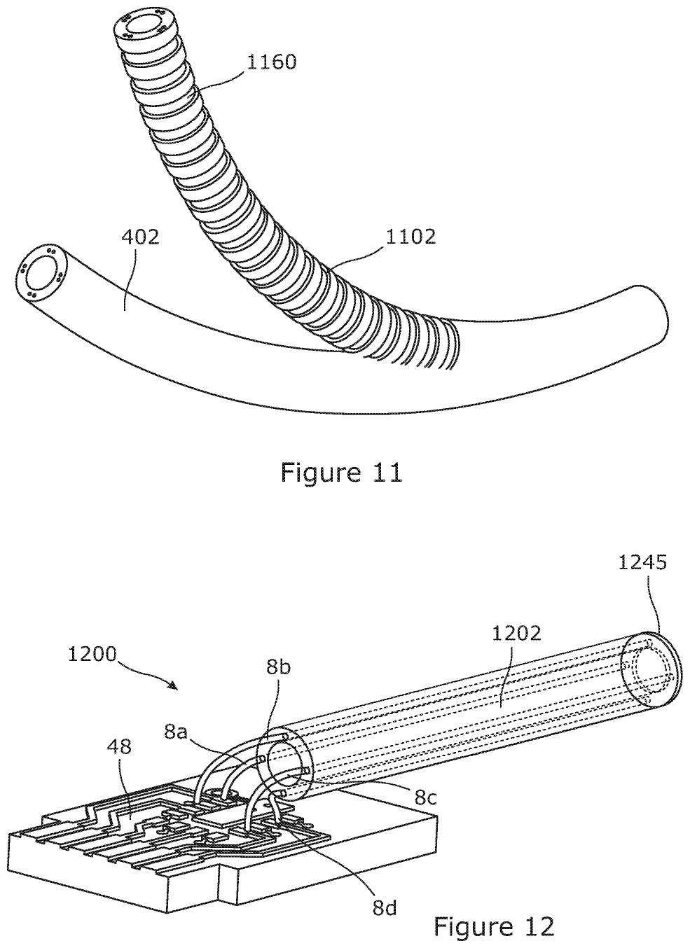

FIG. 11 is schematic representation of a fibre, according to a further embodiment of the first aspect of the invention, that has been laser profiled to provide circumferential grooves.

FIG. 12 is a schematic representation of a fibre, according to a further embodiment of the first aspect of the invention, comprising a plurality of resistance wires connected to a circuit board.

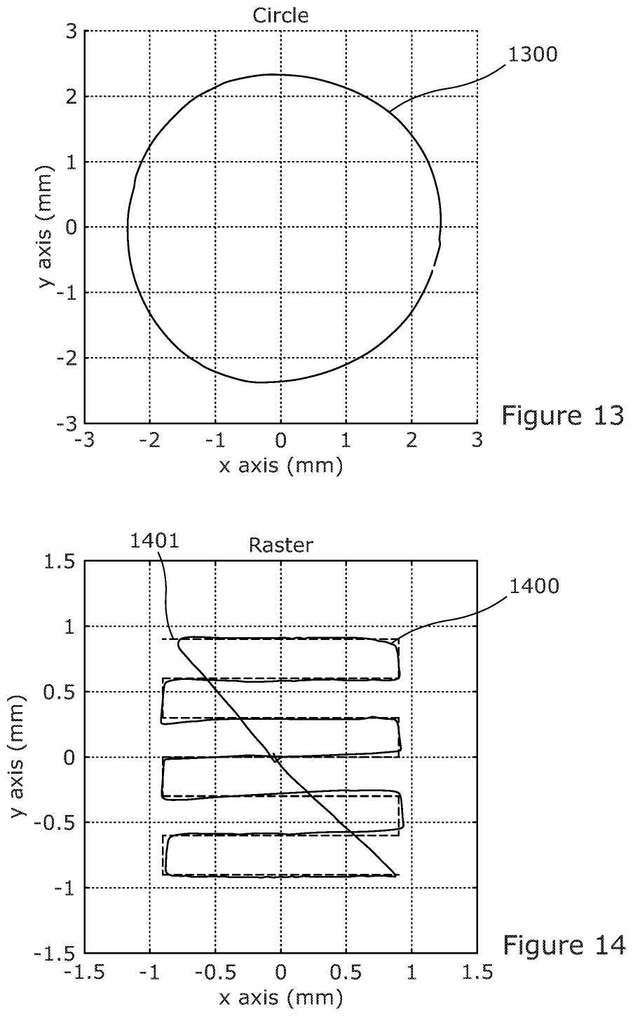

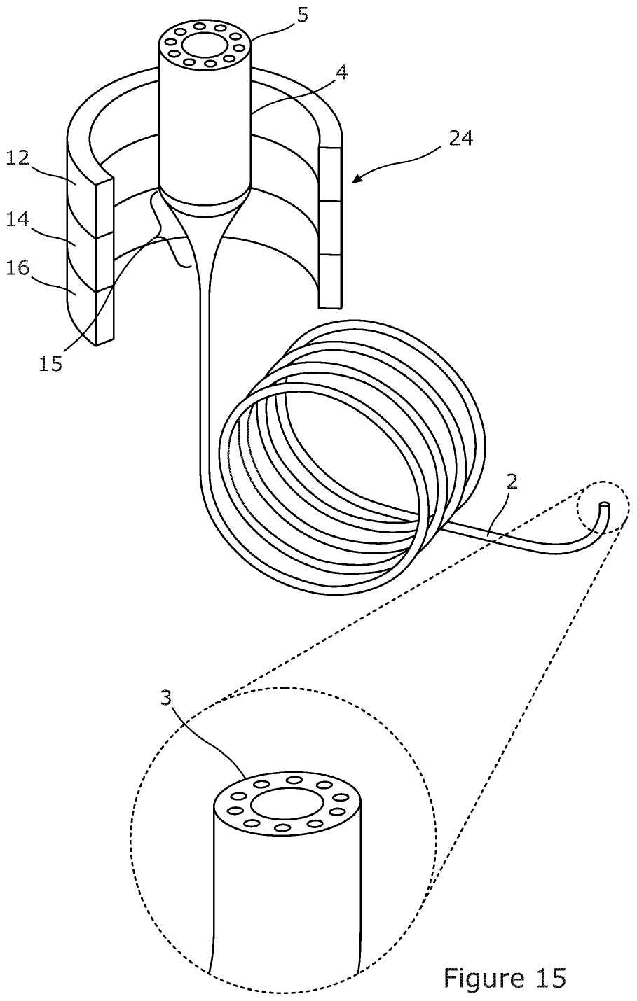

FIGS. 13 and 14 are graphical representations of deflections that may be performed by a fibre according to an embodiment of the first aspect of the invention.

FIG. 15 is a schematic representation of a method drawing a fibre according to an embodiment of the second aspect of the invention.

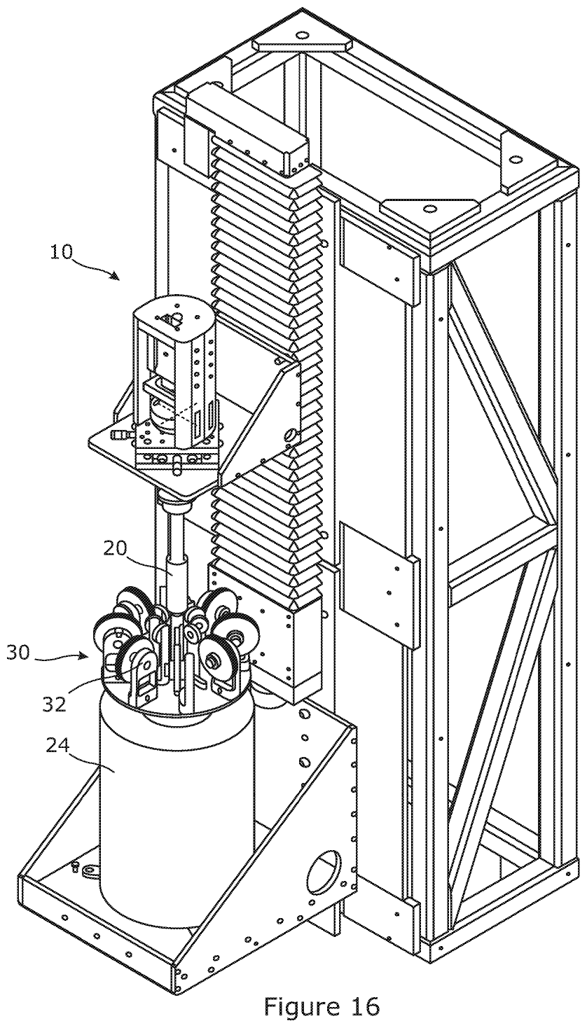

FIG. 16 is a schematic representation of a draw apparatus for carrying out the method shown in FIG. 15.

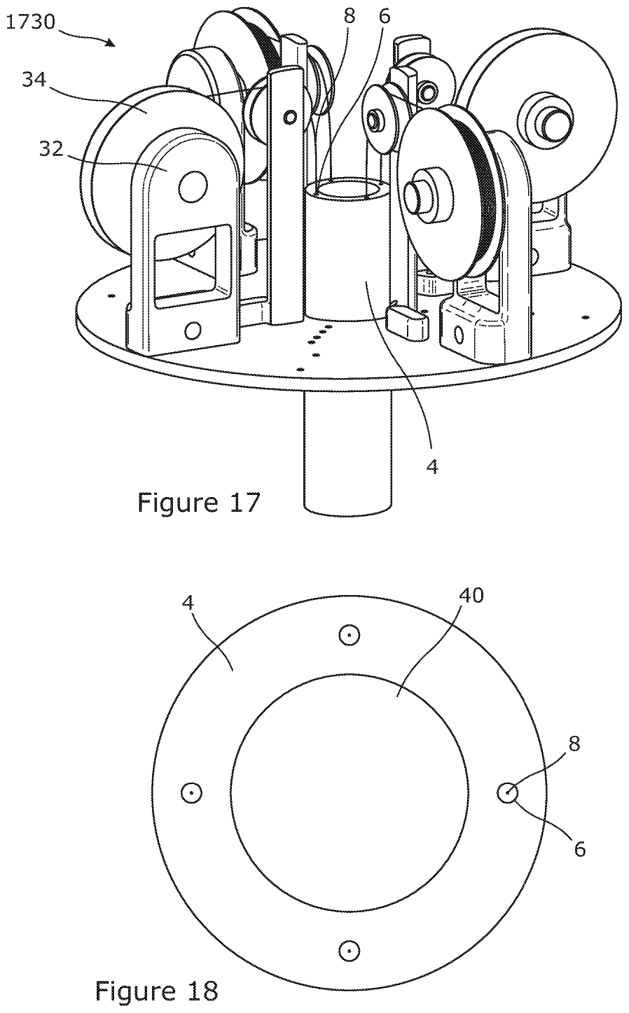

FIG. 17 is a close-up view of a feed apparatus similar to that forming part of the draw apparatus shown in FIG. 16.

FIG. 18 is a cross-sectional schematic representation of a preform to be drawn into a fibre comprising a plurality of resistance wires in accordance with an embodiment of the invention.

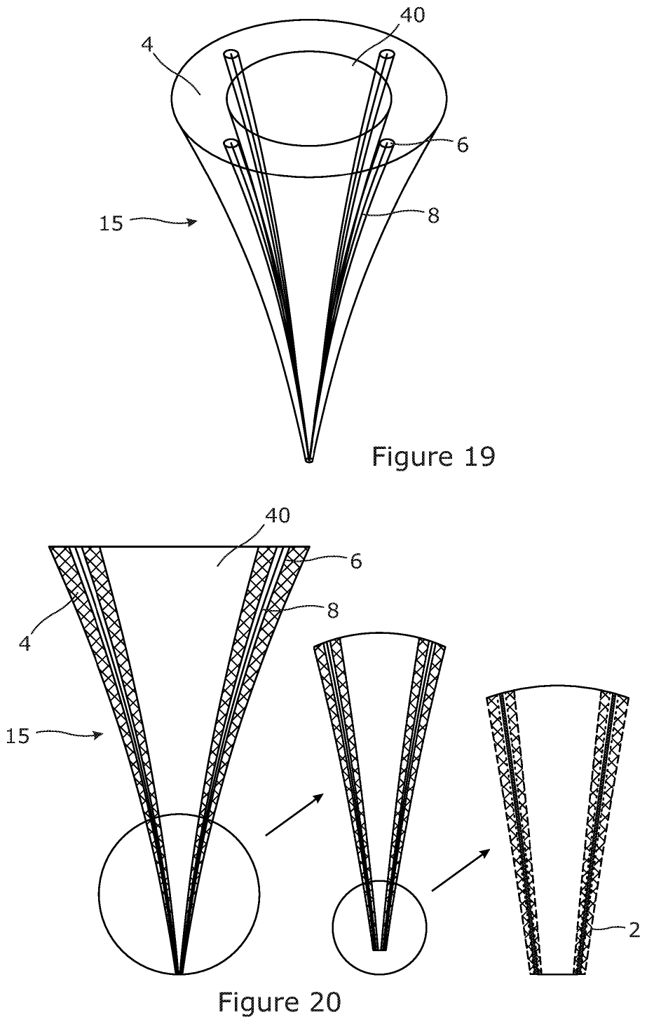

FIG. 19 is a schematic representation of the preform shown in FIG. 18, being drawn to form a fibre.

FIG. 20 is a cross-sectional view of the preform of FIG. 19.

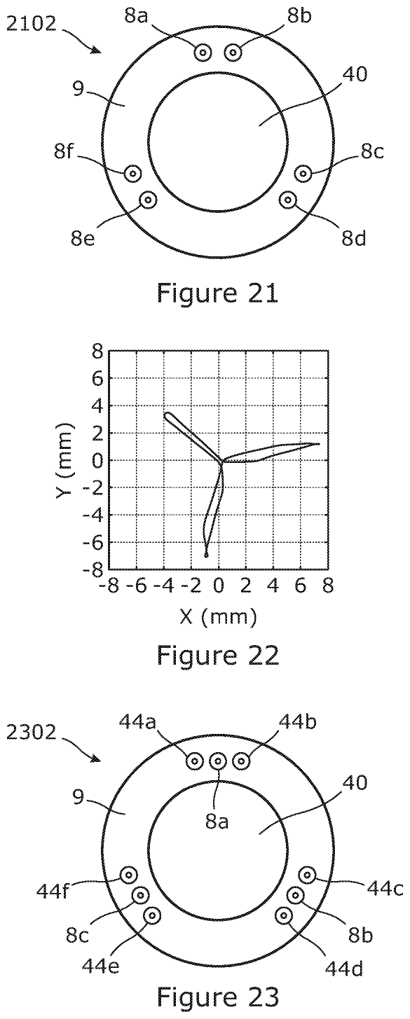

FIG. 21 is a cross-sectional schematic representation of a fibre according to a further embodiment of the first aspect of the invention.

FIGS. 22 is a graphical representation of deflections that may be performed by a fibre according to an embodiment of the first aspect of the invention.

FIG. 23 is a cross-sectional schematic representation of a fibre according to a further embodiment of the first aspect of the invention.

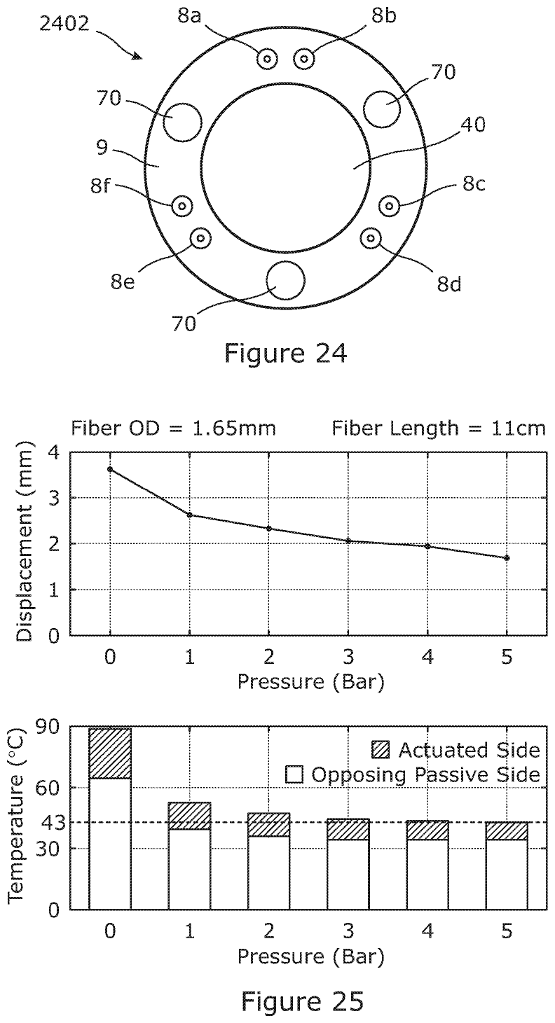

FIG. 24 is a cross-sectional schematic representation of a fibre according to a further embodiment of the first aspect of the invention.

FIG. 25 shows graphical representations of deflections and temperature gradients that may be exhibited by a fibre according to an embodiment of the first aspect of the invention.

Referring initially to FIGS. 1a and 1b, a bendable fibre according to an embodiment of the first aspect of the invention is defined generally by the reference numeral 102. The fibre 102 comprises a resistance wire 8, thermally expandable material 9, a void 40 and a conducting wire 44 spaced slightly apart from the resistance wire 8. In this embodiment of the invention the fibre 102 has a deactivated configuration 150, which is straight and an activated configuration 151, which is curved in the opposite direction of the resistance wire 8.

The resistance wire 8 may have a high electrical resistance meaning that an electrical power applied to the resistance wire 8 may cause it to increase in temperature. When the resistance wire 8 is heated it may cause the thermally expandable material 9 proximal to the resistance wire 8 to also heat such that a temperature gradient forms across the cross-section of the fibre. Due to the material forming the fibre 102 being thermally expandable, the thermally expandable material 9 proximal to the resistance wire 8 may expand while thermally expandable material 9 further away from the resistance wire 8 will expand less or not at all.

In FIG. 1b, the fibre 102 holds the deactivated configuration 150 when power is not supplied to the resistance wire 8. However, when electrical power is supplied to the resistance wire 8, the resistance wire 8 increases in temperature, thereby heating the surrounding thermally expandable material 9 and causing that material to expand. The asymmetric expansion of the fibre 102 causes a deflection of the fibre 102 towards the activated configuration 151. When the power is turned off again, the fibre 102 will return to its deactivated configuration 150. This deflection process is repeatable and is also adjustable based on the amount of electrical power applied to the resistance wire. The greater the electrical power applied, the hotter the resistance wire will get and the greater the degree of deflection that will be caused. This allows precise control of a tip of the fibre without disadvantages associated with known tendon driven devices such as friction and backlash.

The conducting wire 44 may close the electrical circuit required such that electrical current may flow through the resistance wire 8. For example, an electrical power source at a proximal end of the fibre may be connectable to both the resistance wire 8 and the conducting wire 44 while, at a distal end of the fibre, the resistance wire 8 may be connected to the conducting wire 44. Accordingly, electrical current may flow from the electrical power source, along the resistance wire 8 in a first direction through the fibre 102 and then along the conducting wire 44 in a second direction through the fibre 102 opposite to the first direction. In some embodiments of the invention the conducting wire 44 may have a lower resistance than the resistance wire 8 so that only the resistance wire increases substantially in temperature. In other embodiments of the invention the conducting wire 44 may have a resistance similar or equal to the resistance wire 8 so that an electrical current passing through the conducting wire 44 causes it to increase in temperature similarly to the resistance wire 8. This may be beneficial if the resistance wire 8 and the conducting wire 44 are positioned close to one another within the cross-section of the fibre 102, as is the case in FIGS. 1a and 1b, because the deflection of the fibre caused by heat expansion of material close to the wires may be amplified.

The void 40 may extend through part or all of the fibre and may be any suitable shape. In this embodiment of the invention the void 40 is a central lumen with a substantially circular cross-section extending the length of the fibre 102. The void 40 may increase the distance that heat must radiate through the thermally expandable material 9 in order to travel from the resistance wire 8 to an opposite side of fibre 102. The time required for heat to radiate through the fibre 102 is therefore increased such that a greater temperature gradient may be maintained across the cross-section of the fibre. In other words, the void 40 acts to thermally insulate one side of the fibre 102 from the opposite side so that a greater difference in temperature across the cross-section of the fibre 102 may be achieved. The greater the difference in temperature of the thermally expandable material 9 in two opposite parts of the fibre, the greater the resulting deflection of the fibre 102 may be.

In use, the void/central lumen 40 may act as a working channel through which a surgeon may deploy surgical instruments such as probes or catheters. For example, the fibre 102 could form part of an endoscope in which an optical fibre extends through the central lumen 40. In this example, the fibre 102 may be electrothermally actuated to scan an area of in-vivo or ex-vivo tissue sample. The images acquired by the distal lens of the optical fibre may then be mosaiced to build up a larger image of the tissue sample covering the entire scan area. Another example application is rapid evaporative ionisation mass spectrometry (REIMS) in which a surgical laser fibre extends through the central lumen 40 and a suction tube is attached to the fibre 102. In this example, the fibre 102 may be electrothermally actuated to scan the laser over an area of in-vivo or ex-vivo tissue sample. As the laser evaporates the tissue sample, the suction tube would aspirate the aerosol which would then be thermally ionised for analysis by a mass spectrometer to create a data map for the entre scan area.

Referring now to FIGS. 2a and 2b, a fibre 202 according to a further embodiment of the first aspect of the invention is similar to the fibre 102 shown in FIGS. 1a and 1b. However, fibre 202 comprises two resistance wires 8a, 8b positioned in opposite sides of the fibre 202 and two conducting wires 44a, 44b each positioned close to a respective resistance wire 8a, 8b. In this embodiment of the invention each resistance wire 8a, 8b may be caused to increase in temperature, by applying an electrical current, in order to heat and expand the surrounding thermally expandable material 9 of the fibre 202.

In FIG. 2b, the fibre 202 holds a deactivated configuration 250 when power is not supplied to either resistance wire 8a, 8b. However, when power is supplied to one of the resistance wires 8a, the resistance wire 8a increases in temperature, heats the surrounding thermally expandable material 9 and thereby causes expansion of that material surrounding the resistance wire 8a. The partial expansion of the fibre 202 causes a deflection of the fibre 202 towards a first activated configuration 251 (away from the resistance wire 8a). When the power is turned off again, the fibre 202 will return to its neutral configuration 250. Further, when power is supplied to the other of the resistance wires 8b, the resistance wire 8b causes expansion of material on the other side of the fibre 202 and therefore deflection of the fibre 202 towards a first configuration 252 (away from the resistance wire 8b). The deflection processes are each repeatable in any desired sequence, and the degree of deflection in either direction is adjustable based on the amount of electrical power applied to either resistance wire 8a, 8b. The fibre 202 further comprises a first conducting wire 44a and a first conducting wire 44b that may function similarly to the conducting wire 44 shown in FIGS. 8a and 8b with respect to the resistance wires 8a and 8b respectively.

Referring now to FIGS. 3a and 3b, a fibre 302 according to a further embodiment of the first aspect of the invention is similar to the fibre 202 shown in FIGS. 2a and 2b. However, fibre 302 comprises only a single conducting wire 44 which is positioned evenly between the resistance wires 8a, 8b. In this embodiment of the invention the deflection that may be achieved by providing electrical power to each resistance wire is similar to that achieved by the fibre 202 shown in FIG. 2b. However, here each resistance wire 8a, 8b may be connected to the single conducting wire 44 to form parallel circuits that may be activated independently of one another. In such embodiments of the invention it may be beneficial that the conducting wire 44 has a lower resistance than the resistance wires 8a, 8b so that it does not substantially increase in temperature (relative to the resistance wires 8a, 8b) and cause heat expansion that may affect the deflections achieved in either of the activated configurations 351, 352.

Referring now to FIGS. 4a and 4b a fibre 402 according to a further embodiment of the first aspect of the invention is similar to the fibre 202 shown in FIGS. 2a and 2b. However, fibre 402 comprises four resistance wires 8a, 8b, 8c, 8d spaced apart from one another and four conducting wires 44a, 44b, 44c, 44d each positioned close to a respective resistance wire 8a, 8b, 8c, 8d. In this embodiment of the invention each resistance wire 8a, 8b, 8c, 8d may be heated by applying an electrical current similarly to the resistance wires shown in FIGS. 1a to 3b.

In FIG. 4b, the fibre 402 holds a deactivated configuration 450 when power is not supplied to any of the resistance wires 8a, 8b, 8c, 8d. When power is supplied to one of the four resistance wires 8a, 8b, 8c, 8d the selected resistance wire is heated and causes expansion of the material of the fibre 402 which surrounds the selected resistance wire. The fibre 402 is thereby deflected in the direction away from the side of the fibre in which the selected resistance wire is positioned. For example, when power is supplied to only resistance wire 8a, the fibre 402 deflects to a first activated configuration 451. Powering only resistance wire 8b provides a second activated configuration 452, resistance wire 8c a third activated configuration 453 and resistance wire 8d a fourth activated configuration 454.

It is also possible to supply power to two resistance wires at the same time to provide further possible deflections of the fibre 402. For example, when power is supplied to the resistance wires 8a and 8b the fibre 402 deflects to a fifth activated configuration 455. Powering resistance wires 8b and 8c provides a sixth activated configuration 456, resistance wires 8c and 8d a seventh activated configuration 457 and resistance wires 8d and 8a an eighth activated configuration 458.

The deflection processes are each repeatable in any desired sequence and the degree of deflection towards each configuration is adjustable based on the amount of electrical power applied to one or two of the resistance wires 8a, 8b, 8c, 8d. The fibre 402 is therefore capable of a large range of precise deflections.

In use, the fibre 402 may form part of a device according to an embodiment of the first aspect of the invention with a surgical instrument attached to, or extending from, the tip of the fibre 402. The fibre 402 may act as an actuation mechanism to actuate the surgical instrument through a sequence of precise movements, as demonstrated in FIGS. 13 and 14. The fibre 402 has been demonstrated to provide sub-micron precision. The thermal expansion driven actuation may obviate flaws of tendon drive actuation mechanisms such as imprecision caused by friction and/or backlash. Further, the fibre 402 may have smaller cross-sectional dimensions than known tendon driven actuation mechanisms as there is no requirement for tendons, or other moving parts, to extend through the fibre 402. Use of the fibre 402 may therefore be less invasive than use of known actuation mechanisms for surgical instruments. Also, the fibre 402 is bendable and hence more compliant to its surroundings and less likely to cause harm to surrounding soft tissues than known actuation mechanisms with hard surfaces and edges.

The conducting wires 44a, 44b, 44c, 44d may each provide a closed loop for the current to return to an electrical power source having flowed along the associated resistance wire 8a, 8b, 8c, 8d. In some embodiments of the invention the conducting wires 44a, 44b, 44c, 44d may have a lower resistance than the resistance wires 8a, 8b, 8c, 8d so that only the resistance wire increases substantially in temperature. In other embodiments of the invention the conducting wires 44a, 44b, 44c, 44d may have a resistance similar or equal to the resistance wires 8a, 8b, 8c, 8d so that an electrical current passing through one of the conducting wires 44a, 44b, 44c, 44d causes it to increase in temperature similarly to the respective resistance wire 8a, 8b, 8c, 8d.

Referring now to FIGS. 5a and 5b, a fibre 502 according to a further embodiment of the first aspect of the invention is similar to the fibre 402 shown in FIGS. 4a and 4b. However, fibre 502 comprises only a single conducting wire 44. In this embodiment of the invention the deflection that may be achieved by applying a current to each resistance wire is similar to that achieved by the fibre 402 shown in FIG. 4b. However, here each resistance wire 8a, 8b, 8c, 8d may be connected to the single conducting wire 44 similarly to the embodiment of the invention shown in 3a and 3b. An advantage of this embodiment of the invention over that shown in FIGS. 4a and 4b is that fewer wires are required which may reduce manufacturing complexity and cost. However, control of such an embodiment may be more complicated compared to control of embodiments of the invention where each resistance wire has its own associated conducting wire.

Referring now to FIGS. 6a and 6b, a fibre 602 according to a further embodiment of the first aspect of the invention is similar to the fibres 402, 502 shown in FIGS. 4a to 5b. However, fibre 602 does not comprise a conducting wire. Instead, the plurality of resistance wires 8a, 8b, 8c, 8d may be electrically connected to one another at a distal end of the fibre. Therefore, rather than closed circuit loops being formed by a combination of a resistance wire and a conducting wire, they may be formed by a combination of two resistance wires. For example, electrical power may be selectively applied to flow along resistance wires 8a and 8b (the electrical current flowing out along resistance wire 8a and back along resistance wire 8b or vice versa) to cause the fibre 602 to deflect towards a first activated configuration 651 similarly to how the fibres 402 and 502 may be caused to deflect towards their respective fifth activated configurations 455, 555. Accordingly, powering resistance wires 8b and 8c provides a second activated configuration 652, resistance wires 8c and 8d a third activated configuration 653 and resistance wires 8d and 8a a fourth activated configuration 654.

An advantage of this embodiment of the invention is obviating the requirement for one or more conducting wires separate to the resistance wires. Instead, each resistance wire may act both as a resistance wire and a conducting wire. Fewer wires being required may reduce manufacturing complexity and cost. However, the range of control achievable with such an embodiment may be reduced in comparison to the embodiments shown in FIGS. 4a to 5b which have an equivalent number of resistance wires.

Referring now to FIG. 7, a fibre 702 according to a further embodiment of the first aspect of the invention is similar to the fibre 602 except that it comprises and additional four resistance wires 8e, 8f, 8g, 8h. The resistance wires are positioned in pairs: 8a and 8b, 8c and 8d, 8e and 8f, 8g and 8h. In use, power may be provided to a pair of resistance wires simultaneously (with the electrical current flowing out along one wire and back along the other wire) in order to generate a more acute deflection than may be possible if power was provided to only one resistance wire or to two resistance wires spaced further apart. In a sense, one wire in each pair of resistance wires acts as a conducting wire for the other wire in the pair similarly to the embodiment of the invention shown in FIGS. 4a and 4b.

Referring now to FIG. 8, a fibre 802 according to a further embodiment of the first aspect of the invention is similar to the fibre 602 except that it comprises a void 46 between each resistance wire 8a, 8b, 8c, 8d and the outer surface of the fibre 802 in addition to the void 40 extending centrally through the fibre 802. The voids 46 may be considered as air gaps between the resistance wires 8a, 8b, 8c, 8d and the outer surface of the fibre 802 rather than as the central lumen provided by the void 40.

Each void 40, 46 may act as a barrier to heat transmission through the fibre 802. In particular, each void 40, 46 may act to insulate regions of the fibre 802 proximal to the resistance wires 8a, 8b, 8c, 8d, reducing dissipation of heat and causing a greater increase of temperature in the thermally expandable material proximal to the resistance wires 8a, 8b, 8c and 8d. Facilitating a greater temperature increase may also facilitate greater expansion of material and therefore a more acute deflection of the fibre 802. Additionally, the voids/air gaps 46 may act to reduce the amount that the outer surface of the fibre 802 increases when it is deflected, which may be particularly advantageous if the fibre is used in minimally invasive surgery, for example, as the patient may be protected from high temperature material.

Referring now to FIG. 9, a fibre 902 according to a further embodiment of the first aspect of the invention combines the features of the fibre 802 shown in FIG. 8 and the fibre 802 shown in FIG. 8. That is, the fibre 902 comprises eight resistance wires positioned in pairs and voids/air gaps 46 between each pair of resistance wires and the outer surface of the fibre 902. The fibre 902 may therefore benefit from the advantages of both features.

For each of the fibres 602, 702, 802 and 902 a deflection caused by providing power to two resistance wires was simulated. Table 1, below, shows the minimum and maximum temperatures recorded in each fibre, the resulting maximum temperature difference across the cross-section of each fibre and the maximum fibre tip deflection recorded when simulating an actuation of each fibre. The results demonstrate that the incorporation of paired resistance wires in fibre 702 and the incorporation of voids/air gaps 46 in fibre 802 both increase the temperature difference that may be achieved within the fibres, which is representative of a greater temperature gradient across the fibre's cross-section. The greater variation in temperature causes a greater variation in expansion of the thermally expandable material in each fibre and hence a greater maximum fibre tip deflection. The effect is amplified in fibre 902 in which both paired resistance wires and voids/air gaps 46 are incorporated.

TABLE 1
Results from simulating an actuation in the fibres
for a 3 cm long fibre 602, 702, 802 and 902.

| | Minimum | Maximum | Temperature | | Maximum fibre |
|---|---|---|---|---|---|
| Fibre | temperature | temperature | difference | | tip deflection |
| 602 | 57.17° C. | 109.19° C. | 52.02° | C. | 1.3484 mm |
| 702 | 52.51° C. | 131.23° C. | 78.72° | C. | 1.4523 mm |
| 802 | 57.93° C. | 132.99° C. | 75.06° | C. | 1.4521 mm |
| 902 | 45.39° C. | 153.53° C. | 108.14° | C. | 1.5382 mm |

Experimentally, for a longer fibre, a temperature difference of 10° C. across the cross-section of the fibre was sufficient to deflect the fibre tip 4 mm while maintaining the maximum fibre temperature below 70° C.

Referring now to FIGS. 10a and 10b, a fibre 1002, according to a further embodiment of the first aspect of the invention, comprises a resistance wire 8 (e.g. stainless steel wire) similarly to the embodiment shown in FIGS. 1a and 1b. However, rather than comprising a single conducting wire, the fibre 1002 comprises a first conducting wire 1044a (e.g. copper wire), a second conducting wire 1044b (e.g. copper wire) and two electrical connections 1045—one that electrically connects the first conducting wire 1044a to the resistance wire 8 and another that electrically connects the resistance wire 8 to the second conducting wire 1044b. The electrical connections 1045 may be any suitable connector capable of conducting electricity, such as a solder or conductive epoxy/ink paste. Alternatively, the electrical connections 1045 may be formed by exposing the conducting wire 1044a or 1044b and resistance wire 8, twisting the relevant wires together, applying a conductive paint (e.g. silver paint) to the twisted wires and covering the painted wires with a conductive tape (e.g. copper tape). The conductive tape may then optionally be covered with an insulating tape (e.g. Kapton tape) to insulate the electrical connections 1045.

Accordingly an electrical circuit loop may be formed wherein an electrical current may flow along the first conducting wire 1044a in a first direction through the fibre 1002 and then along the resistance wire 1008 and the second conducting wire 1044b in a second direction through the fibre opposite to the first direction, as demonstrated by the electrical current line 1080 in FIG. 10b.

In use, when electrical power is applied to the circuit formed by the resistance wire 1008 and first and second conducting wires 1044a, 1044b, electrical current may flow through a portion of the resistance wire 1008 between the two electrical connections 1045 only. Hence only that portion of the resistance wire will increase in temperature due to its high resistance. Meanwhile, the rest of the resistance wire 1008 will exhibit minimal change in temperature as no electrical current will flow through it and the conducting wires 1044a, 1044b may also show relatively low change in temperature due to having lower electrical resistance. Accordingly, the temperature increase in the fibre 1002 will be localised to the thermally expandable material 9 around the heated portion of the resistance wire 1008 and resulting deflection of the fibre 1002 will be similarly localised.

Referring now to FIG. 11, a fibre 1102, according to a further embodiment of the first aspect of the invention, comprises a plurality of circumferential grooves 1160 positioned incrementally along a portion of the fibre 1102. The circumferential grooves 1160 may be provided by laser profiling the fibre 1102. The circumferential grooves 1160 reduce the stiffness of the fibre 1102 and therefore allow the fibre 1102 to bend further in response to expansion of part of the material forming the fibre 1102. FIG. 11 shows a comparison of the deflection possible with a laser profiled fibre, such as fibre 1102, against the deflection that may be provided by a fibre without laser profiling (such as fibre 402 shown in FIGS. 4a and 4b), despite the same amount of power being supplied.

In other embodiments of the invention, varying the degree of deflection achievable along the length of a fibre may be facilitated in a variety of different ways. For example, in embodiments of the invention, a portion of a resistance wire may comprise an etched surface that increases the electrical resistance through that portion of the fibre. Increasing the electrical resistance increases how hot that portion of the wire will become when electrical power is applied, increases the heating of the surrounding thermally expandable material and causes a greater degree of deflection. In embodiments of the invention, the diameter of the fibre may be varied along its length wherein thinner sections are more flexible and may therefore deflect more than thicker portions. In embodiments of the invention, the fibre may comprise two or more thermally expandable materials with varied material characteristics. For example, a portion of the fibre comprising a softer thermally expandable material will deflect more than a portion comprising a stiffer thermally expandable material. Also, portion of the fibre comprising a thermally expandable material with a greater coefficient of linear thermal expansion will deflect more than a portion comprising a thermally expandable material with a lower coefficient of linear thermal expansion.

Referring now to FIG. 12, a device 1200, according to an embodiment of the first aspect of the invention, comprises the fibre 602 (shown in FIGS. 6a and 6b) wherein the plurality of resistance wires 8a, 8b, 8c, 8d extends from a proximal end of the fibre 602. The device 1200 further comprises a circuit board 48 and an electrical connection 1245 positioned at the distal end of the fibre 602.

Each of the resistance wire 8a, 8b, 8c, 8d extending from the fibre 602 is connected to the circuit board 48 and is also connected to the other resistance wires via the electrical connection 1245, thereby creating a plurality of parallel electrical circuits.

The circuit board 48 may provide digital control of the resistance wires 8, providing electrical power to them in a variety of different combinations to generate desired deflections of the fibre 1202 similar to those shown in FIG. 6b, for example.

The electrical connect 1245 may be any suitable electrically conductive means for connecting the resistance wires 8a, 8b, 8c, 8d, such as solder of conductive ink paste for example.

Referring now to FIGS. 13 and 14, a fibre according to an embodiment of the invention comprising four or more resistance wires, such as any of those shown in FIGS. 4a to 12, may be capable of complex deflections. FIGS. 13 and 14 show simulated movements of a fibre tip caused by sequentially providing power to different combinations of resistance wires. In each Figure, the X and Y axes represent the amount of deflection of the fibre tip from a straight configuration, i.e. coordinates 0,0 would be recorded when the fibre is straight.

In particular FIG. 13 shows a circle 1300 drawn by the fibre tip that is approximately 5 mm in diameter. Meanwhile, FIG. 14 shows a line 1400 drawn by the fibre tip. In this case the fibre tip was actuated and caused to follow a guideline 1401 comprising seven horizontal lines 1.8 mm in length, spaced 0.3 mm apart and joined by vertical lines. FIG. 14 shows that the fibre tip was controlled accurately to within less than 0.1 mm of the guideline 1401.

A fibre capable of such complex and precise actuations may be particularly advantageous in the field of minimally invasive surgery wherein an end effector could be attached to the tip of a fibre and actuated in intricate patterns such as those demonstrated in FIGS. 13 and 14.

Referring now to FIG. 15, a method for drawing a fibre according to the second aspect of the invention is shown wherein the fibre is defined generally by the reference numeral 2. The fibre 2 is drawn from a preform 4 with a preform cross-section 5 wherein the preform is provided to a temperature-controlled apparatus 24 that comprises a pre-heating apparatus 12, a heating apparatus 14 and a quenching apparatus 16. The preform 4 is fed sequentially through the pre-heating apparatus 12 and the heating apparatus 14 in order to raise the temperature of a leading part of the preform 4 and provide a heated portion 15 of the preform 4 that is then suitable to be drawn. The speed of drawing of the fibre 2 may be controlled primarily by gravity or the control of the draw rate may be controlled by a draw apparatus.

In some embodiments of the invention, the preform is initially allowed to neck-down under gravity, after which the tip of the necked-down portion is cut off. Once the necked-down portion has been removed, the remaining drawn fibre may be connected to a capstan which may be used to draw the fibre. Control of the draw speed may be provided by the capstan or may be controlled by any other suitable apparatus. The heated portion 15 of the preform 4 which has been drawn into a fibre 2 is quenched in order to set the fibre shape. Quenching the fibre 2 may be achieved by removing the fibre 2 from the influence of the heating apparatus or, as shown in the example of FIG. 1, the fibre 2 may be cooled slowly to a temperature below the draw temperature by using the quenching apparatus 16. The quenching apparatus 16 may be a heater set to provide a temperature lower than the draw temperature but higher than room temperature, for example.

During the drawing process, the preform cross-section 5 is substantially maintained as the preform 4 transitions to the fibre 2 with a fibre cross-section 3. However the dimensions that traverse the fibre cross-section 3 are significantly smaller than those of the preform cross-section 5. It is therefore possible to provide a preform 4 with a complex cross-sectional design that is relatively straight forward to produce at a large scale and then draw the preform 4 to form the fibre 2 with the same cross-sectional design at a scale that might otherwise be difficult to produce due to its intricacy.

Referring now to FIG. 16, a draw apparatus 10 for drawing a fibre using the method represented in FIG. 1 is shown, wherein the draw apparatus 10 may be known as a draw tower. The draw apparatus 10 comprises a preform mount 20 and a temperature-controlled apparatus 24 that may be equivalent to the temperature-controlled apparatus 24 shown in FIG. 1. The draw apparatus further comprises a wire feed apparatus 30, in this embodiment of the invention the feed apparatus 30 comprises eight resistance wire feeders 32.

In this embodiment of the invention, a preform 4 is mounted in the draw apparatus 10 by way of the preform mount 20, which may be configured to receive preforms of differing sizes. During drawing of the preform 4 into a fibre, the preform 4 is lowered, by the draw apparatus 10, into the temperature-controlled apparatus 24 in order to provide heating and subsequent cooling of the preform 4 and resultant fibre.

Referring now to FIG. 17, a feed apparatus 1730 is similar to, and interchangeable with, the feed apparatus 30 shown in FIG. 16. In this embodiment of the invention the feed apparatus 1730 comprises four resistance wire feeders 32, although it is to be appreciated that a feed apparatus may comprise any suitable number of feeders and the feeders are not limited to being resistance wire feeders only (for example, one or more of the feeders may be conducting wire feeders). Each resistance wire feeder 32 comprises a resistance wire spool 34 that is rotatably attached to the resistance wire feeder 32 and may rotate to dispense a resistance wire 8. Each resistance wire 8 is dispensed and fed into a respective channel 6 of a preform 4. Each resistance wire spool 34 may be driven by a motor to dispense the corresponding resistance wire 8 at the required speed.

FIG. 18 shows a cross-section of the preform 4 which further illustrates each channel 6 and the resistance wire 8 fed into each channel 6. Before the preform 4 is drawn to form a fibre, the diameter of the channels 6 is much larger than the diameter of the resistance wires 8. For example, the channels 6 may have a diameter of 2.5 mm while the resistance wires may have an outer diameter of 50 μm. Hence each resistance wire 8 is loose within its respective channel 6. The preform 4 also comprises a void 40 that extends centrally through the preform 4.

Referring now to FIGS. 19 and 20, a heated portion 15 of a preform 4 is shown being drawn to form a fibre 2. The preform 4, and the channels 6 within it, gradually become narrower in size. The drawing process is continued until a fibre with a desired diameter is formed wherein the diameter of each channel 6 is approximately equal to the outer diameter of each resistance wire, as shown in FIG. 20. For example, the preform may have a diameter of 40 mm and the resultant fibre may have a diameter of 1 mm, meanwhile the diameter of each channel may reduce from 2.5 mm to 50 μm.

Once the fibre 2 is formed, one of the resistance wires 8 may be electrically connected to a voltage power source, causing the resistance wire 8 to be heated. The heated wire may increase the temperature of the surrounding material of the fibre, causing that material to expand. Due to the eccentricity of the resistance wires 8 in the cross-section of the fibre 2, a side of the fibre 2 comprising the heated resistance wire 8 expands while an opposite side of the fibre 2 (not comprising the heated resistance wire 8) maintains its normal length. As a result, the fibre 2 exhibits a deflection in the direction opposite to the expanded part of the fibre 2. The deflection can be varied by activating different combinations of the resistance wires 8, as is shown in the FIGS. 2a to 6b.

It has been found from experiments that when the resistance wires 8 (and conducting wires 44) are fed into the respective channels 6 during the draw, they can sometimes be attached to the expandable material in the formed fibre 2. This has the effect of restricting motion, and therefore displacement of the fibre 2, in subsequent use because the metal typically does not expand as much as the polymer. To address this issue, the diameter of the channels 6 in the preform 4 can be increased so that they have a larger diameter than the wires 8, 44 in the formed fibre 2 and the wires 8, 44 are held more loosely. It is also possible to feed the wires 8, 44 into the respective channels 6 after the preform 4 has been drawn.

FIG. 21 shows a fibre 2102 according to a further embodiment of the first aspect of the invention. This fibre 2102 is similar to those shown in FIGS. 4a and 7 except that it comprises three pairs of wires 8a-f instead of four. Each pair may include two resistance wires 8 as shown, or one resistance wire 8 and one conducting wire 44. By actuating each pair individually or in combination, the use of three pairs of wires 8a-f has been shown to enable displacement of the fibre 2102 in any radial direction. By removing the need for a fourth pair of wires 8g-h, therefore, this fibre 2102 may provide a more cost-effective option from a manufacturing perspective than those shown in FIGS. 4a and 7.

FIG. 22 shows the improved tip displacement that can be achieved when the wires 8, 44 are fed into the respective channels 6 after the preform 4 has been drawn into a fibre 2. This experiment was conducted using a fibre 2102 with the configuration shown in FIG. 21 and demonstrates that displacement of around 7.5 mm in each radial direction is possible.

FIG. 23 shows a fibre 2302 according to another embodiment of the first aspect of the invention. This fibre 2302 is similar to that shown in FIG. 21 except that each pair of wires has been replaced with a set of three wires. As shown, each set of wires comprises first 44a,c,e and second 44b,d,f conducting wires separated by a resistance wire 8a-c as per the embodiment of FIGS. 10a and 10b. This fibre 2302 therefore provides the additional technical advantage of localised heating and deflection.

FIG. 24 shows a fibre 2402 according to another embodiment of the first aspect of the invention. This fibre 2402 is similar to that shown in FIG. 21 except that it further comprises a plurality of voids 70 extending longitudinally at least partially through the fibre 2402. Each void 70 is positioned substantially diametrically opposite a corresponding pair of wires 8a-f and forms a fluid channel along a portion of the fibre 2402. The voids 70 enable a cooling fluid (e.g. a gas such as air, nitrogen or oxygen; or a liquid such as water) to be passed through the thermally expandable material 9 on the opposite side of the central lumen 40 from the corresponding pair of wires 8a-f. This has been found to increase the temperature gradient across the cross-section of the fibre 2402 and thus increase the range of deflection. Furthermore, because each pair of wires 8a-f has a corresponding void 70, the cooling fluid can be applied selectively to increase the range of deflection in any radial direction.

The cooling fluid may additionally or alternatively be passed through the central lumen 40 of the fibre 2402. This has the effect of cooling the thermally expandable material 9 surrounding the central lumen which reduces the surface temperature of the fibre 2402. This may be beneficial for preventing overheating of the thermally expandable material 9 by the resistance wires and also for preventing a patient from being burned by the fibre 2402 during an in-vivo procedure. Cooling the thermally expandable material via the central lumen, however, also reduces the temperature gradient across the cross-section of the fibre 2402 and the associated displacement as described further below.

FIG. 25 shows the results of an experiment in which an air hose was connected to the proximal end of the fibre 2402 by a needle and used to pass cooling air through the central lumen 40 during use of the fibre 2402. In this experiment, the fibre 2402 had a length of 11 cm and a diameter of 1.65 mm, the air was provided from a compressed air supply and a laser displacement sensor was used to measure deflection of the fibre 2402. Furthermore, the experiment was performed within an incubator configured to maintain the temperature of the surrounding environment to 38° C.±1° C., and the pressure inside the fibre 2402 was gradually increased from 0-5 bar using the compressed air.

As illustrated in FIG. 25, the surface temperature on the actuated side of the fibre 2402 (i.e. adjacent the wires 8a-b when viewed in cross-section) decreased from around 90° C. to around 43° C. as the pressure increased, while the surface temperature of the passive side of the fibre 2402 (i.e. adjacent the void 70 when viewed in cross-section) decreased from around 65° C. to around 35° C. as the pressure increased. This represents a decrease in temperature gradient across the cross-section of the fibre 2402 from around 25° C. to around 8° C. as the cooling air was passed through the central lumen. As such, the measured displacement showed a corresponding decrease from around 3.7 mm to around 1.7 mm.

Preferences and options for a given aspect, feature or parameter of the invention should, unless the context indicates otherwise, be regarded as having been disclosed in combination with any and all preferences and options for all other aspects, features and parameters of the invention. For example, it should be regarded that the conducting wire 44 feature shown in FIGS. 1a to 5b has been disclosed in combination with the air gap 46 feature shown in FIGS. 8 and 9. Similarly, the cooling fluid channels 70 shown in FIG. 24 may be applied to the other embodiments of the first aspect of the invention.

1. A device comprising a bendable fibre comprising a fibre axis, a thermally expandable material and a resistance wire extending longitudinally through the fibre spaced apart from the fibre axis; wherein, in use, electrical power applied to the resistance wire causes the temperature of the resistance wire to increase.

2. A device according to claim 1, wherein the thermally expandable material has a coefficient of linear thermal expansion between 0.2×10⁻⁴ and 10×10⁻⁴ K⁻¹.

3. A device according to claim 1, wherein the resistance wire comprises a material having a resistivity between 1×10⁻⁷ and 5×10⁻⁶ Ω·m at 20° C.

4. A device according to claim 1, wherein the fibre further comprises one or more voids extending longitudinally at least partially through the fibre.

5. A device according to claim 4, wherein one of the voids forms a central lumen.

6. A device according to claim 4, wherein one of the voids forms an air gap between the resistance wire and an outer surface of the fibre.

7. A device according to claim 4, wherein one of the voids forms a fluid channel diametrically opposite the resistance wire.

8. A device according to claim 1, further comprising an electrical conductor operatively connected to the resistance wire for applying electrical power to the resistance wire.

9. A device according to claim 8, wherein the electrical conductor comprises a conducting wire extending longitudinally through the fibre spaced apart from the resistance wire, which conducting wire is electrically connectable to the resistance wire.

10. A device according to claim 8, wherein the electrical conductor comprises a first conducting wire and a second conducting wire, each extending longitudinally through the fibre spaced apart from the resistance wire and one another, and the first and second conducting wires are each electrically connectable to the resistance wire.

11. A device according to claim 1, wherein a portion of the resistance wire comprises an etched surface.

12. A device according to claim 1, wherein a portion of the fibre comprises a plurality of circumferential grooves positioned incrementally along the portion.

13. A device according to claim 1, wherein a portion of the fibre has a smaller diameter than the rest of the fibre.

14. A device according to claim 1, wherein the fibre comprises a first portion comprising a first thermally expandable material and a second portion comprising a second thermally expandable material, the first portion being more bendable than the second portion and/or the first thermally expandable material having a higher coefficient of linear thermal expansion than the second thermally expandable material.

15. A device according to claim 1, further comprising a restrictive sheath adapted to receive the at least part of the fibre and restrict deflection of any part of the fibre received within the restrictive sheath.

16. A device according to claim 1, wherein the fibre further comprises a plurality of resistance wires, each extending longitudinally through the fibre and spaced apart from the fibre axis.

17. A method of manufacturing a bendable fibre comprising a fibre axis, thermally expandable material and a resistance wire spaced apart from the fibre axis using a draw apparatus, the method comprising the steps of:
providing a preform comprising a preform axis, a thermally expandable material and a resistance wire channel extending longitudinally through the preform spaced apart from the preform axis, to the draw apparatus;
heating a portion of the preform;
drawing the heated portion of the preform in order to form a fibre; and
feeding a resistance wire into the resistance wire channel during or after the draw such that the resistance wire is positioned within the resistance wire channel of the fibre spaced apart from the fibre axis.

18. A method according to claim 17, wherein the preform further comprises a first conducting wire channel, the method further comprising the further step of:
feeding a first conducting wire into the first conducting wire channel during or after the draw.

19. A method according to claim 18, wherein the preform further comprises a second conducting wire channel, the method further comprising the further step of:
feeding a second conducting wire into the second conducting wire channel during or after the draw.

20. A method according to claim 17 comprising the further step of etching the surface of the resistance wire prior to feeding it into the resistance wire channel.

21. A method according to claim 17 comprising the further step of laser profiling a plurality of circumferential grooves positioned incrementally along a portion of the fibre.

22. A method according to claim 17, wherein the step of drawing the heated portion of the preform comprises varying the speed at which the heated portion of the preform is drawn in order to vary the diameter of the resulting fibre along its length.

23. A method according to claim 17, wherein the preform comprises a plurality of resistance wire channels, and the step of feeding the resistance wire into the resistance wire channel comprises feeding each of the plurality of resistance wires into a respective one of the plurality of resistance wire channels.
